(19) United States
(12) Patent Application Publication
(10) Pub. No.: US 2005/0266578 A1
(43) Pub. Date: Dec. 1, 2005

(54) Methods and systems for detection of macrolides

(76) Inventor: Larry D. Gruenke, Lafayette, CA (US)

Correspondence Address: COZEN O' CONNOR, P.C., 1900 MARKET STREET, PHILADELPHIA, PA, 19103-3508, US

(21) Appl. No.: 11/122,532

(22) Filed: May 5, 2005

Related U.S. Application Data
(60) Provisional application No. 60/568,639, filed on May 6, 2004.

(51) Int. Cl. G01N 33/00

(52) U.S. Cl. 436/94

(57) ABSTRACT

The present invention relates to reverse-phase high performance liquid chromatography (RP-HPLC) methods and systems for detecting macrolides such as erythromycylamine and related compounds.

### CROSS REFERENCE TO RELATED APPLICATIONS

This application claims the benefit of U.S. Ser. No. 60/568,639, filed May 6, 2004, the disclosure of which is incorporated herein by reference in its entirety.

### FIELD OF THE INVENTION

The present invention relates to analytical methods and systems for detecting and quantifying macrolides such as erythromycylamine and related compounds involving reverse-phase high performance liquid chromatography (RP-HPLC) and photometric detection.

### BACKGROUND OF THE INVENTION

Macrolides describe a family of antibiotics used to treat a variety of bacterial infections. Macrolides are characterized chemically by a macrocyclic lactone ring structure of 14 to 16 atoms and usually at least one sugar, amino sugar, or related moiety. Macrolides are believed to inhibit bacterial protein synthesis as a result of binding at two sites on the bacterial 50 S ribosome causing dissociation of transfer RNA and termination of peptide linking. Erythromycin, the first macrolide antibiotic, was discovered in 1952 and entered clinical use shortly thereafter. Erythromycin and the early derivatives (e.g., different salts and esters) are typically characterized by bacteriostatic or bactericidal activity for most gram positive bacteria, in particular streptococci, and good activity for respiratory pathogens. Macrolides proved to be safe and effective for many respiratory infections, and are useful in patients with penicillin allergy.

Macrolides typically have ultraviolet (UV) absorbance in the very low wavelength range (e.g., <220 nm), approaching the limits of photometric detection methods. The United States Pharmacopeia National Formulary (USP-NF) compendial assay method for Erythromycin (see structure below) involves RP-HPLC with L21 stationary phase (reverse-phase, rigid, spherical styrene-divinylbenzene copolymer, 5 to 10 μm particle diameter) using UV detection at 215 nm (see, e.g., pp 663-665 of USP-NF published Jan. 1, 2000). In fact, many reduced Erythromycin derivatives and related molecules such as 9-(S)-erythromycylamine (or PA2794; see structure below) have a UV maximum absorption band (UVₘₐₓ) well below 215 nm. For example, the UVₘₐₓ for 9-(S)-erythromycylamine occurs at about 191 nm, nearing the short wavelength limits of standard photometric detection methods. Accordingly, alternate detection methods such as electrochemical detection have been found attractive (see, e.g., Whitaker, et al., J. Liq. Chromatogr. (1988), 11(14), 3011-20; Pappa-Louisi, et al., J Chromatogr., B: Biomed Sci. Appl. (2001), 755(1-2), 57-64; Kees, et al., J. Chromatogr., A (1998), 812(1+2), 287-293; Hedenmo, et al., J Chromatogr., A (1995), 692(1+2), 161-6; Daszkowski, et al., J. Liq. Chromatogr. Relat. Technol. (1999), 22(5), 641-657; and Dubois, et al., J Chromatogr., B: Biomed. Sci. Appl. (2001), 753(2), 189-202). These alternate methods, however, are usually not suitable for assays that must be run in a Quality Control environment such as assays related to drug release and stability studies. Photometric detection remains the superior method, and most universally accepted method, for purposes of detecting and quantitating assayed compounds such as active pharmaceutical ingredients.

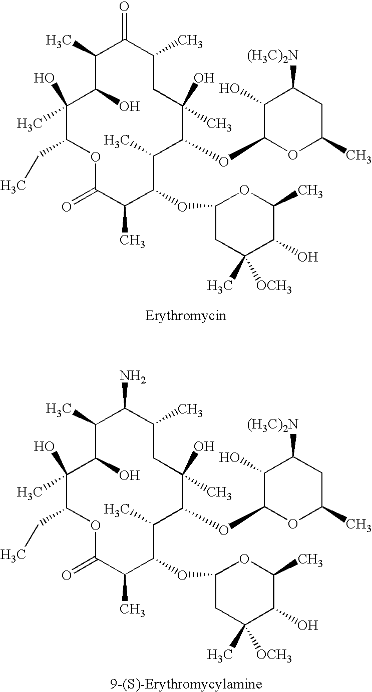

Because photometric detection has numerous apparent advantages over other detection methods when coupled with HPLC analysis, there is a current need for assays that are suitably designed to detect and quantify low wavelength-absorbing molecules such as macrolide antibiotics using UV detection methods. The methods and systems described herein help meet this and other needs.

### SUMMARY OF THE INVENTION

The present invention provides a method of detecting a macrolide having maximum absorption in the ultraviolet-visible range at about 180 nm to about 220 nm, comprising:
a) applying a sample containing said macrolide to a reverse-phase high performance liquid chromatography (RP-HPLC) column outfitted with an ultraviolet (UV) detector having a detection wavelength between about 180 nm and about 220 nm;
b) eluting said sample with a mobile phase comprising an ion pair reagent; and
c) monitoring column effluent with the UV detector to detect an absorption or transmission peak at the detection wavelength, where the peak corresponds to the macrolide.

In some embodiments, the macrolide has maximum absorption in the ultraviolet-visible range at about 180 nm to about 200 nm. In further embodiments, the macrolide is 9-(S)-erythromycylamine.

In some embodiments, the detection wavelength is about 190 nm to about 210 nm, about 197 nm to about 205 nm, or about 200 nm.

In some embodiments, the ion pair reagent comprises a C₄-C₁₂ alkylsulfonate salt such as sodium 1-octane sulfonate. In further embodiments, the concentration of ion pair reagent in the mobile phase is about 10 mM to about 30 mM.

In some embodiments, the mobile phase further comprises a buffer such as a sulfate or phosphate buffer. In further embodiments, the mobile phase has a pH of about 1 to about 4, such as about 3.

In some embodiments, the mobile phase comprises variable amounts of water, organic solvent, and buffer over the time course of elution of the macrolide. In further embodiments, the mobile phase maintains substantially constant absorbance at the detection wavelength over the time course of elution.

In some embodiments, the mobile phase has negligible absorbance above about 205 nm. In further embodiments, the mobile phase has an absorbance of less than about 0.5 at 200 nm. In yet further embodiments, the mobile phase has an absorbance of less than about 0.1 at 200 nm.

In some embodiments, the methods of the invention further comprise quantifying the macrolide by comparing peak area corresponding to the macrolide with a standard.

In some embodiments, the methods of the invention further comprise detecting impurities present in the sample by monitoring column effluent with the UV detector to detect one or more further absorption or transmission peaks at the detection wavelength, where the one or more further peaks has a peak area greater than about 0.05% of the peak area for the macrolide and corresponds to one or more impurities.

In some embodiments, the present invention further provides a method of determining purity of a sample by: i) monitoring column effluent with the UV detector to detect one or more further absorption or transmission peaks at the detection wavelength, where the one or more further peaks corresponds to one or more impurities; and ii) measuring characteristics of the peaks detected by the detector to calculate impurity content in the sample.

The present invention further provides a method of detecting 9-(S)-erythromycylamine comprising:
a) applying a sample containing said 9-(S)-erythromycylamine on a C18 reverse-phase high performance liquid chromatography (RP-HPLC) column outfitted with an ultraviolet (UV) detector having a detection wavelength of about 200 nm;
b) eluting said sample with mobile phase comprising water, acetonitrile, sodium 1-octane sulfonate and a sulfate buffer; and
c) monitoring column effluent with said UV detector to detect an absorption or transmission peak at the detection wavelength, said peak corresponding to said 9-(S)-erythromycylamine.

The present invention further provides a system for detecting a macrolide, comprising:
a) a reverse phase high performance liquid chromatography column (RP-HPLC) comprising:
   i) a stationary phase comprising reverse phase solid support;
   ii) a mobile phase comprising:
      1) an ion pair reagent;
      2) buffer;
      3) water; and
      4) acetonitrile, wherein the mobile phase has less than about 0.5 absorbance at 200 nm; and
b) an ultraviolet (UV) detector.

### BRIEF DESCRIPTION OF THE DRAWINGS

FIG. 1 shows an example chromatogram for an RP-HPLC assay of 9-(S)-erythromycylamine run under the conditions described in Example 1.

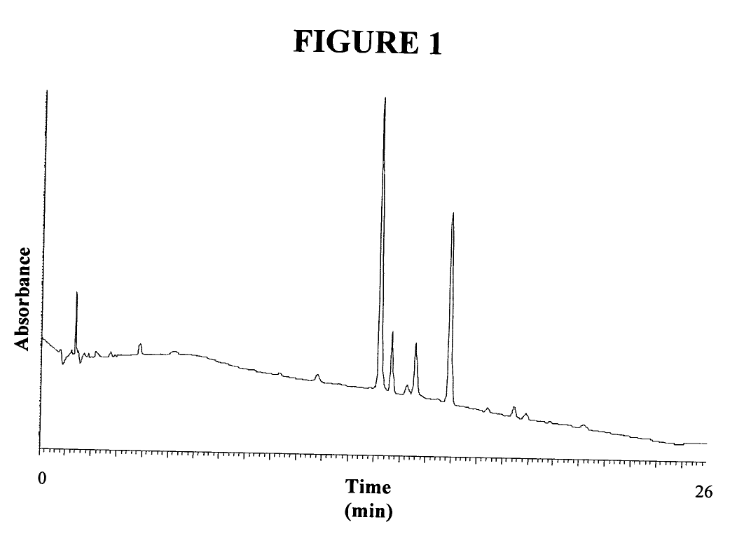

FIG. 2 shows the effect of ion pair reagent (sodium 1-octanesulfonate) concentration in the mobile phase on an RP-HPLC assay of 9-(S)-erythromycylamine according to the invention. The central part of the chromatogram is shown to demonstrate, for example, the effect of assay conditions on peak separation of the major components.

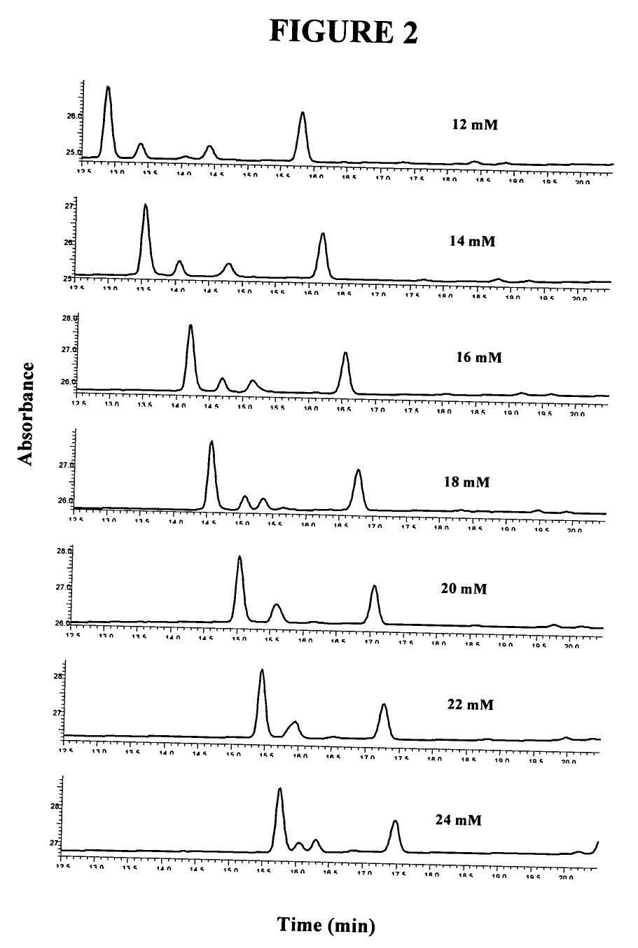

FIG. 3 shows the effect of mobile phase pH on the RP-HPLC assay of 9-(S)-erythromycylamine according to the invention.

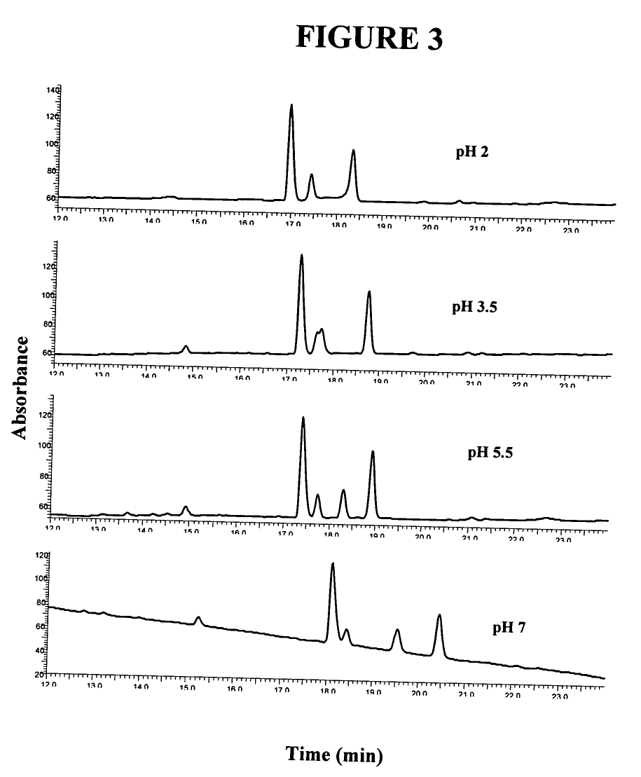

### DETAILED DESCRIPTION

The present invention provides, inter alia, methods and systems for the detection and quantitation of macrolides and/or related molecules having primary spectrophotometric absorption in the UV region. In some embodiments, the methods involve running a sample containing the macrolide or related compound on a reverse-phase high performance liquid chromatography (RP-HPLC) column. The sample is eluted with a low UV-absorbing mobile phase to allow detection of the macrolide and/or any separated impurities at short wavelengths. The mobile phase can also contain an ion pair reagent to improve retention of the often polar macrolides on the reverse-phase column.

Macrolides according to the present invention include any of the known antibiotic or other macrolides and their derivatives. Typical macrolides are characterized by a 12-, 14-, or 16-membered macrocyclic lactone core structure. Macrolides are widely known in the art and are thoroughly described in, for example, Macrolide Antibiotics, ed. Satoshi Omura, Academic Press, Inc., Orlando, Fla., 1984, which is incorporated herein by reference in its entirety. In some embodiments, the macrolide has a relatively poor ultraviolet-visible (UV-VIS) absorption profile, for example, showing maximum absorption in the UV-VIS range (about 100 nm to about 900 nm) at a wavelength of about 180 nm to about 220 nm, about 180 nm to about 200 nm, or about 180 μm to about 195 nm. In further embodiments, the macrolide has a maximum absorption in the UV-VIS range at a wavelength of about 188, about 189, about 190, about 191, about 192, about 193, about 194, about 195, about 196, about 197, about 198, about 199, about 200, about 201, about 202, about 203, about 204, or about 205 nm. Macrolides suitable for detection by the methods and systems described herein can further include at least one moiety that can be readily protonated or deprotonated to form a charged macrolide capable of pairing with an ion pair reagent. In some embodiments, the macrolide can contain a neutral, basic moiety where the corresponding free moiety (where H takes the place of the remainder of the macrolide molecule) has a pKb below about 10, below about 8, below about 5, or below about 4. Some exemplary neutral, basic moieties include NH2, alkylamines (e.g., NHMe, NHEt, etc.), dialkylamines (e.g., NMe₂, NEt₂, etc.), cyclic amines (e.g., piperidinyl, morpholino, etc.), and the like.

Representative macrolides that can be detected by the methods and systems provided herein include, for example, 9-(S)-erythromycylamine, 9-(R)-erythromycylamine, erythromycin, erythromycin hydrazone, erythromycin, 9-imino erythromycin, erythromycin oxime, erythromycin B, erythromycin hydrazone B, 9-imino erythromycin B, erythromycylamine B, erythromycin hydrazone acetone aduct, 9-hydroxyimino erythromycin, erythromycylamine hydroxide, 9-hydroxyimino erythromycin B, erythromycylamine B hydroxide, erythromycylamine C, erythromycylamine D, azithromycin, clarithromycin, dirithromycin, troleandomycin, derivatives thereof and the like.

Further suitable macrolides contain no more than one sp or sp2 hybridized carbon or nitrogen atoms. Examples of macrolides characterized as such include erythromycylamine, erythromycylamine B, erythromycylamine hydroxide, erythromycylamine B hydroxide, erythromycylamine C, erythromycylamine C hydroxide, erythromycylamine D, erythromycylamine D hydroxide, and the like.

In some embodiments, the macrolide is 9-(S)-erythromycylamine.

The mobile phase according to the invention can be any combination of liquid components that effectively elutes the desired macrolide, allows for separation of the macrolide from potential impurities, and allows photometric detection of the macrolide at the detection wavelength. In some embodiments, the mobile phase has negligible absorbance (e.g., measured with a spectrophotometer over a 1 cm pathlength) above about 205 nm. By “negligible” is meant absorbance of about 0.02 or less. In further embodiments, the mobile phase has an absorbance (e.g., measured with a spectrophotometer over a 1 cm pathlength) of less than about 0.5, less than about 0.3, or less than about 0.1 at the detection wavelength.

The mobile phase can contain water, organic solvent, or a mixture thereof. Any suitable organic solvent that is miscible with water and does not interfere with detection of the macrolide at the detection wavelength can be used. In some embodiments, the organic solvent is acetonitrile. The mobile phase can contain 0 to 100% (v/v) water and 0 to 100% (v/v) organic solvent. In some embodiments, the mobile phase contains about 5 to about 75, about 10 to about 60, or about 20 to about 50% (v/v) organic solvent.

In some embodiments, the mobile phase further includes a buffer to stabilize the solution at a desired pH. Any buffer that does not interfere with the detection of the macrolide at the detection wavelength can be used. In some embodiments, the buffer is a phosphate or sulfate buffer. In further embodiments, the buffer is a sulfate buffer. Buffer concentration can be, for example, about 0.1 mM to about 1000 mM. In some embodiments, buffer concentration is about 1 mM to about 500 mM, about 5 mM to about 100 mM, or about 10 mM to about 30 mM.

The mobile phase can have any pH at which the macrolide is sufficiently stable such that it can be detected by the methods and systems of the invention. In some embodiments, the pH is about 1 to about 4. In further embodiments, the pH is about 3.

In some embodiments, the mobile phase includes an ion pair reagent, such as for example, a salt that facilitates retention of the macrolide on a reverse-phase column. Any ion pair reagent that is reasonably stable in the mobile phase solution, is capable of forming an ion pair with a charged form (e.g., protonated or deprotonated) of the macrolide, and does not interfere with elution or detection of the macrolide is suitable. Various suitable ion pair reagents are commercially available and HPLC techniques using the same are well known in the art. Some example ion pair reagents include alkylsulfonate salts such as (C₄-C₁₂ alkyl)sulfonate salts including sodium 1-octanesulfonate. The concentration of ion pair reagent in the mobile phase can be about 0.1 mM to about 1000 mM. In some further embodiments, ion pair concentration is about 1 mM to about 500 mM, about 5 mM to about 100 mM, or about 10 mM to about 30 mM. In some embodiments, the ion pair concentration is about 12 mM to about 15 mM.

The mobile phase can be run through the HPLC column as an isocratic elution or gradient elution. In embodiments where a gradient mobile phase is applied, the mobile phase can be comprised of a mixture of two or more different eluent solutions, the proportions of which vary over the time course of the elution. For example, the mobile phase can contain variable amounts of water, organic solvent, buffer, and ion pair reagent during elution. The variation in component amounts can be adjusted such that the gradient mobile phase maintains substantially constant absorbance at the detection wavelength during the course of elution. The variation in component amounts can also be adjusted to optimize peak shape, elution time, separation of macrolide from impurities, and other parameters.

In some embodiments, the mobile phase composition is varied by eluting with one or a mixture of two eluent solutions, each containing different amounts of water, organic solvent, buffer, and ion pair reagent. In some embodiments, a first eluent solution contains about 10 to about 30% (v/v) organic solvent, about 70 to about 90% (v/v) water, about 10 to about 20 mM ion pair reagent, and about 10 to about 15 mM buffer and a second eluent solution contains about 40 to about 60% (v/v) organic solvent, about 40 to about 60% (v/v) water, about 8 to about 15 mM ion pair reagent, and about 8 to about 12 mM buffer. In further embodiments, a first eluent solution contains about 20% (v/v) organic solvent, about 80% (v/v) water, about 15 mM ion pair reagent, and about 13 mM buffer and a second eluent solution contains about 50% (v/v) organic solvent, about 50% (v/v) water, about 12 mM ion pair reagent, and about 10.5 mM buffer. At any point during elution, the mobile phase can be composed of 100% of one of the two eluent solutions or a mixture of the two.

The stationary phase can be composed of any reverse-phase solid support medium that in combination with the mobile phase allows for the detection of the macrolide and separation of the same from potential impurities. In some embodiments, the stationary phase contains a C8 to C18 matrix. In further embodiments, the stationary phase is a C18 matrix.

The sample can be diluted to form an diluted sample for introduction into the column. The diluted sample can have a macrolide concentration of about 1 to about 10 mg/mL. Sample diluent can be comprised of water buffered by Bis-Tris (e.g., about 20 to about 100 mM, about 30 to about 70 mM, or about 50 mM of Bis-Tris) and having a pH of about 6 to about 8, or about 7.

The UV detector monitoring effluent from the column can include any spectrophotometer capable of detecting absorption or transmission of UV wavelengths through a liquid sample. The detector can be tuned to a detection wavelength which can be constant for the duration of elution. In some embodiments, effluent is monitored at a wavelength of about 190 nm to about 210 nm, about 197 nm to about 205 nm, or about 200 nim. In some embodiments, the detection wavelength is about 200 nm.

Elution of the macrolide according to the methods and systems of the invention can be carried out under a variety of temperatures and pressures, including ambient temperature and pressure. In some embodiments, elution is carried out at a constant temperature of about 10 to about 45, 10 to about 30, about 15 to about 25, or about 20° C. Temperature can be maintained below room temperature by, for example, outfitting the column with a chiller. Elution can also be carried out under air or an inert atmosphere.

Detection of the macrolide can be confirmed by comparing a chromatogram believed to contain a peak corresponding to the macrolide with a chromatogram run under the same conditions showing a peak for a known sample of the macrolide. For example, a sample peak appearing within about 0.2 min of the reference peak can be considered confirmed. The amount of macrolide in a sample can also be quantified by comparing the area of a peak corresponding to the macrolide with the area of a peak in a chromatogram obtained for a reference sample (standard) containing a known amount of the macrolide.

The present invention further provides a method of determining purity of a sample containing a macrolide by detecting the macrolide according to the detection methods described herein and monitoring column effluent with the UV detector to detect one or more further absorption or transmission peaks at the detection wavelength, where the further peak or peaks correspond to compounds other than the detected macrolide such as impurities (for example, other macrolides) present in the sample. In some embodiments, peaks corresponding to impurities can be recognized as having peak areas greater than about 0.05% of the peak area for the detected macrolide. Further, sample purity can be assessed by measuring peak characteristics, including for example, peak height and/or peak area. For example, purity can be assessed by determining percentage of total peak area (e.g., peak area of macrolide plus peak areas of impurities) for each detected impurity.

Also encompassed by the invention are systems including an assembly of the components described above. For example, a system of the invention can contain a) a reverse phase high performance liquid chromatography column (RP-HPLC) containing i) stationary phase comprising reverse phase solid support matrix; and ii) a mobile phase as described above; and b) an ultraviolet (UV) detector.

Additional parameters for running and optimizing an HPLC assay according to the present invention are well within the knowledge of one skilled in the art as evidenced in the literature, for example, by Snyder et al., Practical HPLC Method Development, 2^{nd} ed., Wiley, New York, 1997, the disclosure of which is incorporated herein by reference in its entirety.

The invention is described in greater detail by way of specific examples. The following examples are offered for illustrative purposes, and are not intended to limit the invention in any manner. Those of skill in the art will readily recognize a variety of noncritical parameters which can be changed or modified to yield essentially the same results.

### EXAMPLES

### Example 1

HPLC Assay for Detection of Erythromycylamine and Determination of Purity

General Description

A test sample was prepared for HPLC analysis by dissolution of 9-(S)-erythromycylamine (manufactured by Alembic Ltd., India) in 50 mM Bis-Tris buffer. Bis-Tris was used to stabilize impurities commonly found in samples of 9-(S)-erythromycylamine. HPLC analysis of this sample solution on a reverse phase column was accomplished by using sodium 1-octanesulfonate in the mobile phase as an ion pairing agent. Components were detected by UV absorption at 200 nm. The presence of 9-(S)-erythromycylamine in the sample was confirmed if a peak was seen in the chromatogram of the sample that was within ±0.2 min of the main peak seen in the chromatogram of the reference standard. The absence of 9-(S)-erythromycylamine was confirmed if a peak was not seen in the sample chromatogram within ±0.2 min of the main peak seen in the chromatogram of the reference standard. The 9-(S)-erythromycylamine peak area in the sample chromatogram was compared to that from a 9-(S)-erythromycylamine reference solution chromatogram to determine the 9-(S)-erythromycylamine percent weight/weight assay value. Impurities were reported by retention time and percent total area in the chromatogram.

Instrumentation

The following equipment operated according the manufacturer's instructions was used for obtaining HPLC chromatograms. Equipment with comparable performance can be substituted.
Vacuum Degasser: Waters 2690
Pump: Waters 2690
Injector: Waters 2690
   50:50 mixture by volume of acetonitrile and water as needle wash
   100 μL injection loop
   Pre-column: Phenomenex Security Guard with ODS cartridge (P/N AJO-4287)
   Column: Phenomenex Column, C18(2), 150 mm×4.6 mm, 5 μm (P/N 00F-4252-E0)
   Column was installed in the direction of the eluent flow as instructed on the column label and placed in a Jones Chromatography column chiller/heater maintained at 20±1° C.
   Column was stored in 70:30 (v/v) acetonitrile:water when not in use.
   Detector: Waters 2487 Dual Wavelength Detector
   Wavelength=200 nm
   Column Chiller: Jones Chromatography model 7955
   Temperature was set to 20±1° C.
   Data System: Perkin-Elmer Nelson Turbochrom data system, version 6.2.1
      Preparation of Test Samples and Reference Solutions

Test sample and reference solutions were not stored for more than 36 hours.

Test Sample: 9-(S)-Erythromycylamine API (active pharmaceutical ingredient) test sample solution was prepared by weighing, in duplicate, 60±6 mg of the test sample into 25-mL volumetric flasks. Weights were recorded to the nearest 0.01 mg. 20 mL of Sample Diluent B (see below) were added to each flask. The samples were sonicated until completely dissolved. Ice was added to the sonication bath to keep the temperature below 15° C. The solution was made up to the 25 mL volume by addition of Sample Diluent B (see below).

Reference Standard Solution: 9-(S)-Erythromycylamine reference standard was prepared by weighing, in duplicate, 60±6 mg of the 9-(S)-erythromycylamine reference standard into 25-mL volumetric flasks. Weights were recorded to the nearest 0.01 mg. 20 mL of Sample Diluent B (see below) were added to each flask. The samples were sonicated until completely dissolved. Ice was added to the sonication bath to keep the temperature below 15° C. The solution was made up to the 25 mL volume by addition of Sample Diluent B (see below). One flask was labeled #1 and the other #2.

Sensitivity Standard Solution: The sensitivity standard solution was prepared by pipetting 100.0 μL of the reference standard solution #1 into a 100 mL volumetric flask that was made to volume with Sample Diluent A.

Working Solutions

Sample Diluent A:

50 mM Bis-Tris (Sigma)
pH 7.4.
   Sample Diluent B:
50 mM Bis-Tris (Sigma)
pH 7.0.
   Eluent A:
20% acetonitrile (HPLC grade, Fisher)
15 mM sodium 1-octanesulfonte (Fluka)
13 mM Na₂SO₄ (Sigma)
pH 3.1
   Eluent B:
50% acetonitrile (HPLC grade, Fisher)
12 mM sodium 1-octanesulfonte (Fluka)
10.5 mM Na₂SO₄ (Sigma)
pH 3.1
   HPLC Analysis

Sample analysis was carried out under the following parameters.
Flow rate: 1.0 mL/min
Injection Volume: 20 μL
Run Time: 40 min
Wavelength: 200 nm

The data system was set to acquire 1 point/second with a 40 min acquisition time.

Blanks, reference standards, and test samples were injected in the order according to Table I below.

TABLE I

| Injection | Test |
|---|---|
| Blanks | System equilibration and |
| | Acceptability of reagents |
| Ref. Stand. #1 | Instrument precision, |
| | Calibration & acceptability of column |
| Blank | System equilibration |
| Sens. Standard | Assay sensitivity |
| Ref. Stand. #2 | Accuracy, verification of standard preparation |
| Test Samples | Sample analysis |
| Ref. Stand. #1 | Calibration and agreement of standards |
| Blank | carryover |

A gradient mobile phase was applied for each run according to Table II below.

TABLE II

| Time (min) | % Eluent A (v/v) | % Eluent B (v/v) |
|---|---|---|
| 0 | 70 | 30 |
| 1 | 70 | 30 |
| 20 | 30 | 70 |
| 26 | 30 | 70 |
| 27 | 0 | 100 |
| 31 | 0 | 100 |
| 33 | 70 | 30 |
| 40 | 70 | 30 |

Confirmation of 9-(S)-Erythromycylamine Presence in Sample

The presence of 9-(S)-erythromycylamine was confirmed by determination that the principal peak of the chromatogram was within 0.2 min of the principal peak in the Reference Standard Solution chromatogram.

Calculation of Impurity Content

Chromatograms of duplicate preparations were examined and peak areas integrated and averaged. Impurity content (purity) was calculated as follows:
Impurity Content=(Aᵢ×100)/(Σ[Aᵢ]+A_{P})
where:
Aᵢ is the area of the impurity peak;
A_{P} is the area of the 9-(S)-erythromycylamine peak; and

Σ[Aᵢ] is the sum of the areas of all impurity peaks greater than 0.05%.

### Example 2

Effect of Detection Wavelength Variation on % Peak Areas for HPLC analysis of 9-(S)-Erythromycylamine

Tables IIIa-IIIc compare HPLC data at UV detection wavelengths of 197, 200, and 203 nm for 9-(S)-erythromycylamine samples. The assays were run according to the procedure of Example 1 with the exception of differing detection wavelength (Tables IIIa and IIIc). Relative retention times of 0.94 and 1.24 were observed as having a double peak. The relative retention time (Rel. R.T.) of 9-(S)-erythromycylamine was set to 1.00.

TABLE IIIa
% Peak Area by Relative Retention Time at Detection Wavelength 197 nm

| Rel. | | | | | | | | | | | | | | |
|---|---|---|---|---|---|---|---|---|---|---|---|---|---|---|
| R.T | 0.34 | 0.65 | 0.79 | 0.84 | 0.89 | 0.94 | 1.00 | 1.11 | 1.15 | 1.24 | 1.46 | 1.74 | 1.78 | 1.91 |
| Run 1 | 0.04 | 0.09 | 1.31 | 0.42 | 0.12 | 0.60 | 95.22 | 0.11 | 0.37 | 0.82 | 0.03 | 0.14 | — | 0.05 |
| Run 2 | — | 0.11 | 1.25 | 0.36 | 0.12 | 0.58 | 95.72 | 0.22 | 0.39 | 0.92 | — | 0.18 | 0.14 | 0.06 |
| Run 3 | 0.04 | 0.08 | 1.36 | 0.48 | 0.16 | 0.62 | 95.25 | 0.09 | 0.36 | 0.69 | 0.07 | 0.14 | 0.15 | 0.06 |
| Avg | 0.04 | 0.09 | 1.31 | 0.42 | 0.13 | 0.60 | 95.40 | 0.14 | 0.37 | 0.81 | — | 0.12 | 0.15 | 0.06 |
| STD | 0.00 | 0.02 | 0.06 | 0.06 | 0.02 | 0.02 | 0.28 | 0.07 | 0.02 | 0.12 | — | 0.03 | 0.01 | 0.01 |

TABLE IIIb
% Peak Area by Relative Retention Time at Detection Wavelength 200 nm

| Rel. | | | | | | | | | | | | | | |
|---|---|---|---|---|---|---|---|---|---|---|---|---|---|---|
| R.T | 0.34 | 0.65 | 0.79 | 0.84 | 0.89 | 0.94 | 1.00 | 1.11 | 1.15 | 1.24 | 1.46 | 1.74 | 1.78 | 1.91 |
| Run 6 | 0.06 | 0.06 | 1.26 | 0.4 | 0.16 | 0.86 | 95.38 | 0.06 | 0.35 | 0.87 | 0.04 | 0.06 | 0.13 | 0.07 |
| Run 7 | 0.05 | 0.07 | 1.25 | 0.42 | 0.13 | 0.85 | 95.27 | 0.11 | 0.39 | 0.90 | 0.06 | 0.07 | 0.15 | 0.06 |
| Run 8 | 0.06 | 0.06 | 1.26 | 0.44 | 0.17 | 0.81 | 95.48 | 0.05 | 0.30 | 0.86 | 0.08 | 0.07 | 0.13 | 0.07 |
| Avg | 0.06 | 0.06 | 1.26 | 0.42 | 0.15 | 0.84 | 95.38 | 0.07 | 0.35 | 0.88 | 0.06 | 0.07 | 0.14 | 0.07 |
| STD | 0.01 | 0.01 | 0.01 | 0.02 | 0.02 | 0.03 | 0.11 | 0.03 | 0.05 | 0.02 | 0.02 | 0.01 | 0.01 | 0.01 |

TABLE IIIc
% Peak Area by Relative Retention Time at Detection Wavelength 203 nm

| Rel. | | | | | | | | | | | | | | |
|---|---|---|---|---|---|---|---|---|---|---|---|---|---|---|
| R.T | 0.34 | 0.65 | 0.79 | 0.84 | 0.89 | 0.94 | 1.00 | 1.11 | 1.15 | 1.24 | 1.46 | 1.74 | 1.78 | 1.91 |
| Run 9 | 0.08 | 0.07 | 1.27 | 0.45 | — | 0.93 | 95.34 | 0.08 | 0.37 | 1.03 | 0.07 | 0.05 | 0.18 | — |
| Run 10 | 0.08 | 0.06 | 1.25 | 0.52 | 0.19 | 1.01 | 94.86 | 0.15 | 0.43 | 1.10 | 0.08 | — | 0.17 | — |
| Run 11 | 0.08 | 0.07 | 1.25 | 0.48 | 0.12 | 0.93 | 95.14 | 0.07 | 0.21 | 1.01 | 0.08 | 0.10 | 0.45 | — |
| Avg | 0.08 | 0.07 | 1.26 | 0.48 | 0.16 | 0.96 | 95.11 | 0.10 | 0.34 | 1.05 | 0.08 | 0.08 | 0.27 | — |
| STD | 0.00 | 0.01 | 0.01 | 0.04 | 0.05 | 0.05 | 0.24 | 0.04 | 0.11 | 0.05 | 0.01 | 0.04 | 0.16 | — |

The percent areas of the impurity peaks with relative retention times of 0.94 and 1.24 did show some dependency on wavelength; however, the variation in the detection wavelength had little effect on the estimation of most impurities.

### Example 3

Effect of Ion Pair Reagent Concentration on % Peak Areas for HPLC analysis of 9-(S)-Erythromycylamine

Tables IVa-IVe compare HPLC data at sodium 1-octanesulfonate concentrations of 15/12, 14/11, 16/13, 16/11, 14/13 mM (Eluent A/Eluent B). The assays were run according to the procedure of Example 1 with the exception of differing concentrations of ion pair reagent. The relative retention time (Rel. R.T.) of 9-(S)-erythromycylamine was set to 1.00. The notation “dp” refers to observation of a double peak and “sh” refers to observation of a shoulder.

TABLE IVa
% Peak Area by Relative Retention Time at Ion Pair Conc. of 15/12 mM

| Rel. | | | | | | | | | | | | | | |
|---|---|---|---|---|---|---|---|---|---|---|---|---|---|---|
| R.T | 0.33 | 0.64 | 0.79 | 0.84 | 0.89 | 0.94 sh | 1.00 | 1.10 | 1.15 | 1.25 | 1.44 | 1.73 | 1.77 | 1.91 |
| Run 12 | 0.05 | 0.07 | 1.25 | 0.40 | 0.16 | 0.77 | 95.27 | 0.06 | 0.35 | 0.87 | 0.06 | 0.10 | 0.13 | 0.06 |
| Run 13 | 0.05 | 0.07 | 1.25 | 0.40 | 0.13 | 0.76 | 95.25 | 0.11 | 0.39 | 0.87 | 0.06 | 0.11 | 0.15 | — |
| Run 14 | 0.05 | 0.06 | 1.26 | 0.43 | 0.17 | 0.79 | 95.49 | 0.06 | 0.33 | 0.84 | 0.08 | — | 0.07 | — |
| Avg | 0.05 | 0.07 | 1.25 | 0.41 | 0.15 | 0.77 | 95.34 | 0.08 | 0.36 | 0.86 | 0.07 | 0.11 | 0.12 | 0.06 |
| STD | 0.00 | 0.01 | 0.01 | 0.02 | 0.02 | 0.02 | 0.13 | 0.03 | 0.03 | 0.02 | 0.01 | 0.01 | 0.04 | — |

TABLE IVb
% Peak Area by Relative Retention Time at Ion Pair Conc. of 14/11 mM

| Rel. | | | | | | | | | | | | | | |
|---|---|---|---|---|---|---|---|---|---|---|---|---|---|---|
| R.T | 0.34 | 0.66 | 0.79 | 0.85 | 0.90 | 0.95 | 1.00 | 1.12 | 1.16 | 1.24 dp | 1.46 | 1.73 | 1.78 | 1.90 |
| Run 15 | 0.06 | 0.08 | 1.31 | 0.44 | 0.13 | 0.73 | 95.63 | 0.08 | 0.21 | 0.89 | 0.05 | 0.07 | 0.11 | 0.10 |
| Run 16 | 0.07 | 0.08 | 1.27 | 0.46 | 0.15 | 0.77 | 95.26 | 0.15 | 0.32 | 0.96 | 0.08 | 0.07 | 0.11 | 0.12 |
| Run 17 | 0.06 | 0.07 | 1.28 | 0.47 | 0.13 | 0.72 | 95.17 | 0.10 | 0.44 | 1.05 | 0.08 | 0.11 | 0.11 | 0.09 |
| Avg | 0.06 | 0.08 | 1.29 | 0.46 | 0.14 | 0.74 | 95.35 | 0.11 | 0.32 | 0.97 | 0.07 | 0.08 | 0.11 | 0.10 |
| STD | 0.01 | 0.01 | 0.02 | 0.02 | 0.01 | 0.03 | 0.24 | 0.04 | 0.12 | 0.08 | 0.02 | 0.02 | 0.00 | 0.02 |

TABLE IVc
% Peak Area by Relative Retention Time at Ion Pair Conc. of 16/13 mM

| Rel. | | | | | | | | | | | | | |
|---|---|---|---|---|---|---|---|---|---|---|---|---|---|
| R.T | 0.33 | 0.62 | 0.79 | 0.83 | 0.88 | 0.93 dp | 1.00 | 1.09 | 1.15 | 1.24 | 1.43 | 1.72 | 1.77 |
| Run 18 | 0.05 | 0.06 | 1.32 | 0.48 | 0.13 | 0.78 | 95.31 | 0.10 | 0.33 | 0.92 | 0.07 | 0.05 | 0.17 |
| Run 19 | 0.05 | 0.07 | 1.23 | 0.36 | 0.10 | 0.80 | 95.65 | 0.16 | 0.37 | 0.96 | 0.06 | 0.05 | 0.15 |
| Run 20 | 0.05 | 0.09 | 1.31 | 0.48 | 0.16 | 0.84 | 95.36 | 0.08 | 0.35 | 0.94 | 0.06 | — | 0.16 |
| Avg | 0.05 | 0.07 | 1.29 | 0.44 | 0.13 | 0.81 | 95.44 | 0.11 | 0.35 | 0.94 | 0.06 | 0.05 | 0.16 |
| STD | 0.00 | 0.02 | 0.05 | 0.07 | 0.03 | 0.03 | 0.18 | 0.04 | 0.02 | 0.02 | 0.01 | 0.00 | 0.01 |

TABLE IVd
% Peak Area by Relative Retention Time at Ion Pair Conc. of 16/11 mM

| Rel. | | | | | | | | | | | | | | |
|---|---|---|---|---|---|---|---|---|---|---|---|---|---|---|
| R.T | 0.33 | 0.64 | 0.79 | 0.84 | 0.89 | 0.94 sh | 1.00 | 1.10 | 1.15 | 1.25 | 1.44 | 1.71 | 1.76 | 1.89 |
| Run 21 | 0.05 | 0.08 | 1.27 | 0.49 | 0.12 | 0.80 | 95.07 | 0.14 | 0.43 | 1.00 | 0.07 | 0.09 | 0.16 | 0.06 |
| Run 22 | 0.05 | 0.08 | 1.27 | 0.48 | 0.13 | 0.81 | 95.19 | 0.10 | 0.38 | 1.01 | 0.07 | 0.06 | 0.15 | 0.07 |
| Run 23 | 0.04 | 0.07 | 1.25 | 0.47 | 0.11 | 0.81 | 95.20 | 0.13 | 0.39 | 0.99 | 0.06 | 0.05 | 0.16 | 0.05 |
| Avg | 0.05 | 0.08 | 1.26 | 0.48 | 0.12 | 0.81 | 95.15 | 0.12 | 0.40 | 1.00 | 0.07 | 0.07 | 0.16 | 0.06 |
| STD | 0.01 | 0.01 | 0.01 | 0.01 | 0.01 | 0.01 | 0.07 | 0.02 | 0.03 | 0.01 | 0.01 | 0.02 | 0.01 | 0.01 |

TABLE IVe
% Peak Area by Relative Retention Time at Ion Pair Conc. of 14/13 mM

| Rel. | | | | | | | | | | | | | | |
|---|---|---|---|---|---|---|---|---|---|---|---|---|---|---|
| R.T | 0.34 | 0.64 | 0.79 | 0.84 | 0.89 | 0.94 sh | 1.00 | 1.10 | 1.16 | 1.25 | 1.44 | 1.73 | 1.78 | 1.92 |
| Run 24 | 0.06 | 0.10 | 1.28 | 0.45 | 0.14 | 0.82 | 95.45 | 0.05 | 0.34 | 0.93 | 0.07 | 0.05 | 0.15 | 0.06 |
| Run 25 | 0.07 | 0.05 | 1.27 | 0.42 | 0.08 | 0.73 | 95.50 | 0.07 | 0.30 | 1.02 | 0.07 | 0.07 | 0.15 | 0.08 |
| Run 26 | 0.06 | 0.08 | 1.27 | 0.42 | 0.09 | 0.79 | 95.62 | 0.07 | 0.34 | 0.95 | — | 0.06 | 0.14 | 0.07 |
| Avg | 0.06 | 0.08 | 1.27 | 0.43 | 0.10 | 0.78 | 95.52 | 0.06 | 0.33 | 0.97 | 0.07 | 0.06 | 0.15 | 0.07 |
| STD | 0.01 | 0.03 | 0.01 | 0.02 | 0.03 | 0.05 | 0.09 | 0.01 | 0.02 | 0.05 | 0.00 | 0.01 | 0.01 | 0.01 |

In general, retention times increased or decreased when the average ion pair reagent concentration throughout the run increased or decreased. Changing ion pair reagent concentration in opposite directions caused the baseline to slope up or down. With the 16/13 mM combination of mobile phase concentrations, retention time of the slowest eluting impurity peak was extended so much that is was not reliably detected. Otherwise, relative retention times and peak areas were consistent under all conditions tested. In some instances, partially fused peaks were seen to resolve or coalesce as the mobile phase composition was changed.

### Example 4

Effect of pH on % Peak Areas for HPLC analysis of Erythromycylamine

Tables Va-Vc compare HPLC data at pHs of 2.9, 3.1 and 3.3. The assays were run according to the procedure of Example 1 with the exception of differing pH. The relative retention time (Rel. R.T.) of 9-(S)-erythromycylamine was set to 1.00. The notation “dp” refers to observation of a double peak and “sh” refers to observation of a shoulder. No effect of pH was observed on peak areas.

TABLE Va
% Peak Area by Relative Retention Time at pH 2.9

| Rel. | | | | | | | | | | | | | | |
|---|---|---|---|---|---|---|---|---|---|---|---|---|---|---|
| R.T | 0.30 | 0.64 | 0.80 | 0.83 | 0.89 | 0.93 dp | 1.00 | 1.08 | 1.14 | 1.21 | 1.39 | 1.64 | 1.68 | 1.79 |
| Run 27 | 0.06 | — | 1.30 | 0.50 | 0.14 | 0.83 | 95.04 | 0.04 | 0.31 | 0.78 | 0.05 | 0.08 | 0.11 | 0.06 |
| Run 28 | 0.06 | 0.02 | 1.32 | 0.48 | 0.12 | 0.75 | 95.38 | 0.03 | 0.31 | 0.78 | 0.05 | 0.06 | 0.15 | 0.07 |
| Run 29 | 0.07 | 0.02 | 1.28 | 0.46 | 0.12 | 0.73 | 94.73 | 0.04 | 0.28 | 0.79 | 0.06 | — | 0.11 | 0.06 |
| Avg | 0.06 | 0.02 | 1.30 | 0.48 | 0.13 | 0.77 | 95.05 | 0.04 | 0.30 | 0.78 | 0.05 | 0.07 | 0.12 | 0.06 |
| STD | 0.01 | 0.00 | 0.02 | 0.02 | 0.01 | 0.05 | 0.33 | 0.01 | 0.02 | 0.01 | 0.01 | 0.01 | 0.02 | 0.01 |

TABLE Vb
% Peak Area by Relative Retention Time at pH 3.1

| Rel. | | | | | | | | | | | | | | |
|---|---|---|---|---|---|---|---|---|---|---|---|---|---|---|
| R.T | 0.30 | 0.64 | 0.80 | 0.83 | 0.89 | 0.93 dp | 1.00 | 1.09 | 1.14 | 1.22 | 1.40 | 1.65 | 1.69 | 1.80 |
| Run 30 | 0.06 | 0.08 | 1.29 | 0.58 | 0.17 | 0.93 | 94.28 | 0.08 | 0.34 | 1.00 | 0.06 | 0.07 | 0.18 | 0.08 |
| Run 31 | 0.08 | 0.06 | 1.30 | 0.59 | 0.14 | 0.87 | 94.24 | 0.15 | 0.38 | 1.02 | 0.11 | 0.05 | 0.17 | 0.09 |
| Run 32 | 0.06 | 0.04 | 1.27 | 0.45 | 0.07 | 0.76 | 94.79 | 0.08 | 0.36 | 1.04 | 0.05 | 0.05 | 0.17 | 0.07 |
| Avg | 0.07 | 0.06 | 1.29 | 0.54 | 0.13 | 0.85 | 94.44 | 0.10 | 0.36 | 1.02 | 0.07 | 0.06 | 0.17 | 0.08 |
| STD | 0.01 | 0.02 | 0.02 | 0.08 | 0.05 | 0.09 | 0.31 | 0.04 | 0.02 | 0.02 | 0.03 | 0.01 | 0.01 | 0.01 |

TABLE Vc
% Peak Area by Relative Retention Time at pH 3.3

| Rel. | | | | | | 0.93 | | | | | | | | |
|---|---|---|---|---|---|---|---|---|---|---|---|---|---|---|
| R.T | 0.30 | 0.64 | 0.80 | 0.83 | 0.88 | dp | 1.00 | 1.09 | 1.12 | 1.22 | 1.39 | 1.64 | 1.68 | 1.80 |
| Run 33 | 0.04 | 0.06 | 1.37 | 0.61 | 0.17 | 0.84 | 94.17 | 0.08 | 0.30 | 0.99 | 0.07 | 0.05 | 0.18 | 0.14 |
| Run 34 | 0.04 | 0.06 | 1.33 | 0.58 | 0.14 | 0.81 | 94.72 | 0.09 | 0.38 | 0.96 | 0.06 | 0.06 | 0.12 | 0.03 |
| Run 35 | 0.04 | 0.02 | 1.33 | 0.60 | 0.10 | 0.87 | 94.59 | 0.05 | 0.22 | 0.98 | 0.07 | 0.06 | 0.19 | 0.03 |
| Avg | 0.04 | 0.05 | 1.34 | 0.60 | 0.14 | 0.84 | 94.49 | 0.07 | 0.30 | 0.98 | 0.07 | 0.06 | 0.16 | 0.07 |
| STD | 0.00 | 0.02 | 0.02 | 0.02 | 0.04 | 0.03 | 0.29 | 0.02 | 0.08 | 0.02 | 0.01 | 0.01 | 0.04 | 0.06 |

### Example 5

Effect of Column Temperature % Peak Areas for HPLC analysis of 9-(S)-Erythromycylamine

Test assays of 9-(S)-erythromycylamine were run according to the procedure of Example 1 with the exception of differing column temperatures (18, 20, and 22° C.). No significant temperature effects were observed for peak areas over this temperature range. However, lowering of column temperature led to better peak separation and narrowing of the 9-(S)-erythromycylamine peak.

### Example 6

Effect of Solution Concentrations on % Peak Areas for HPLC analysis of 9-(S)-Erythromycylamine

Test assays of 9-(S)-erythromycylamine were run according to the procedure of Example 1 with the exception of differing sample concentrations (0.9, 1.2, and 1.4 mg/mL) of 9-(S)-erythromycylamine. Sample concentration over this range had no significant effect on the quantitative estimation of 9-(S)-erythromycylamine.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable subcombination.

Various modifications of the invention, in addition to those described herein, will be apparent to those skilled in the art from the foregoing description. Such modifications are also intended to fall within the scope of the appended claims. Each reference cited in the present application is incorporated herein by reference in its entirety.

1. A method of detecting a macrolide having maximum absorption in the ultraviolet-visible range at about 180 nm to about 220 nm, comprising:
a) applying a sample containing said macrolide to a reverse-phase high performance liquid chromatography (RP-HPLC) column outfitted with an ultraviolet (UV) detector having a detection wavelength between about 180 nm and about 220 nm;
b) eluting said sample with a mobile phase comprising an ion pair reagent; and
c) monitoring column effluent with said UV detector to detect an absorption or transmission peak at said detection wavelength, said peak corresponding to said macrolide.

2. The method of claim 1 wherein said macrolide has maximum absorption in the ultraviolet-visible range at about 180 nm to about 200 nm.

3. The method of claim 1 wherein said macrolide is 9-(S)-erythromycylamine.

4. The method of claim 1 wherein said detection wavelength is about 190 nm to about 210 mm.

5. The method of claim 1 wherein said detection wavelength is about 197 rn to about 205 nm.

6. The method of claim 1 wherein said ion pair reagent comprises a (C₄-C₁₂ alkyl)sulfonate salt.

7. The method of claim 1 wherein said ion pair reagent comprises sodium 1-octane sulfonate.

8. The method of claim 1 wherein said mobile phase further comprises a buffer.

9. The method of claim 8 wherein said buffer is a phosphate or sulfate buffer

10. The method of claim 1 wherein said mobile phase has a pH of about 1 to about 4.

11. The method of claim 1 wherein said mobile phase maintains substantially constant absorbance at the detection wavelength of said UV detector over the time course of elution of said macrolide.

12. The method of claim 11 wherein said mobile phase has negligible absorbance above about 205 nm.

13. The method of claim 1 further comprising quantifying said macrolide by comparing the area of said peak with a standard.

14. The method of claim 1 further comprising detecting impurities present in said sample by monitoring column effluent with said UV detector to detect one or more further absorption or transmission peaks at said detection wavelength, said one or more further peaks having peak areas greater than about 0.05% of the peak area for said macrolide and corresponding to one or more impurities.

15. The method of claim 1 further comprising determining purity of said sample by:
i) monitoring column effluent with said UV detector to detect one or more further absorption or transmission peaks at said detection wavelength, said one or more further peaks corresponding to one or more impurities; and
ii) measuring characteristics of the peaks detected by said detector to calculate impurity content in said sample.

16. The method of claim 15 wherein said characteristics are peak area or peak height.

17. The method of claim 15 wherein said measuring is carried out by i) determining peak area for each detected impurity and said macrolide; and ii) calculating the percentage of total peak area due to said macrolide.

18. The method of claim 15 wherein each of said one or more further absorption or transmission peaks has a peak area greater than about 0.05% of the peak area for said macrolide.

19. A method of detecting 9-(S)-erythromycylamine comprising:
a) applying a sample containing said 9-(S)-erythromycylamine on a C18 reverse-phase high performance liquid chromatography (RP-HPLC) column outfitted with an ultraviolet (UV) detector having a detection wavelength of about 200 nm;
b) eluting said sample with mobile phase comprising water, acetonitrile, sodium 1-octane sulfonate and a sulfate buffer; and
c) monitoring column effluent with said UV detector to detect an absorption or transmission peak at the detection wavelength, said peak corresponding to said 9-(S)-erythromycylamine.

20. A system for detecting a macrolide, comprising:
a) a reverse phase high performance liquid chromatography column (RP-HPLC) comprising:
i) a stationary phase comprising reverse-phase solid support;
ii) a mobile phase comprising:
1) an ion pair reagent;
2) buffer;
3) water; and
4) acetonitrile, wherein said mobile phase has less than about 0.5 absorbance at 200 nm; and
b) an ultraviolet (UV) detector.
